(19) United States
(12) Patent Application Publication
(10) Pub. No.: US 2006/0272679 A1
(43) Pub. Date: Dec. 7, 2006

(54) Method and apparatus for pretreating filter media prior to installation of the filter media in a filter

(76) Inventors: R. Lee Roberts, Chadds Ford, PA (US); Mark Kevin Addison, Bear, DE (US)

Correspondence Address: MEREK, BLACKMON & VOORHEES, LLC, 673 S. WASHINGTON ST., ALEXANDRIA, WV, 22314, US

(21) Appl. No.: 11/144,837

(22) Filed: Jun. 6, 2005

(51) Int. Cl. B08B 7/04; B08B 9/20; B08B 3/00

(52) U.S. Cl. 134/10; 134/25.1; 134/110

(57) ABSTRACT

A pretreatment unit and associated method for pre-treating filter media used to filter water or wastewater prior to installation in a filter. Preferably, the pretreatment unit includes a pretreatment chamber for temporarily storing and pre-treating filter media prior to installation of the filter media in a filter. The pretreatment chamber has a liquid distribution system for distributing liquid through the filter media at a sufficient velocity to remove fines or other unwanted material from the filter media. The washing liquid may be super chlorinated to sterilize the filter media. The pretreatment unit also includes a collector for collecting fines or other unwanted material removed from the filter media. Preferably, at least a portion of the collector is disposed in the pretreatment chamber. The pretreatment unit further includes a filter media discharge port for discharging filter media treated in the pretreatment chamber. The preferred method of pre-treating filter media is designed to eliminate or significantly reduce the costly and arduous conventional treatment procedures performed on filter media after the filter media is installed in a filter.

### FIELD OF THE INVENTION

The present invention is directed to a method and apparatus for pre-treating filter media prior to installation of the filter media in a filter. The pretreatment process is designed to remove fines or other unwanted material and/or sterilize the filter media prior to installation of the filter media in a filter.

### BACKGROUND OF THE INVENTION

Granular filter media is employed in a variety of known filters for filtering water or wastewater. The granular filter media is typically produced from naturally occurring materials such as anthracite, granite, silica sand, ilmenite and glauconite. Granular filter media formed from naturally occurring materials is often contaminated with unwanted foreign materials including sticks, leaves, dirt, debris and other foreign substances. Also, the granular filter material includes fines, i.e., particles that are smaller than the desired size or size range. Failure to remove the aforementioned unwanted foreign materials including the fines from the filter media forming the filter bed of a filter results in the following drawbacks, among others: (i) a significant reduction in the length that the filter can be run between washing operations; (ii) increased headloss across the filter bed; (iii) turbidity breakthrough: (iv) the inability of the filter including the filter bed to meet project specifications; and/or (v) difficulty in sterilizing the filter media.

Current methodologies for removing fines and other unwanted foreign material from the granular filter media include screening the filter media in either a wet or dry process. Devices such as cyclones have also been used in an attempt to separate the granular filter media from unwanted foreign materials. These methodologies are inadequate for a number of reasons including but not limited to the fact that objectionable material often still remains in the filter media after shipment to the job site despite attempts to remove these undesired materials.

The current practice to remove unwanted foreign materials remaining in the filter media requires installation of the filter media in the filter and subsequent processing of the filter media prior to putting the filter in service, i.e., prior to operating the filter in the filtration mode. Processing of the filter media includes washing or backwashing filter media installed in the filter by directing a liquid upwardly through the filter bed at a velocity sufficient to fluidize the filter media so that the fines rise to the top of the filter bed once the flow of washing liquid is interrupted. Typically, this washing or backwashing process is performed with water only for approximately 5 to 15 minutes. After the media is sufficiently washed or backwashed, the liquid flow is interrupted and the water in the filter is drained. Subsequently, the upper portion of the filter bed is skimmed off and discarded in an attempt to remove the unwanted fines from the filter bed. Typically, the top 1/16 inch to the top ½ inch of the filter bed is removed during the skimming process. The skimming process is usually performed with a rake, a board, a shovel or other suitable implement. The backwashing and skimming procedures are typically repeated three or more times to remove the fines from a given lot of filter media. For filter beds utilizing multiple layers of media, for example, anthracite over sand, each layer of media is repeatedly backwashed and skimmed prior to installation of the next layer.

Once all of the media is installed and prepared as described above, the filter is sterilized as required by most regulatory agencies. The sterilization process is typically accomplished by filling the filter with water, dissolving a significant amount of chlorine or other suitable disinfectant into the water, and maintaining a disinfectant residual for at least 24 hours until the disinfectant demand is stabilized. In cases with high organic contamination, the sterilization phase can take as long as a month for the disinfectant demand to be stabilized. Thus, the filter is unable to be placed in service for prolonged periods of time, resulting in loss of production.

### OBJECTS AND SUMMARY OF THE INVENTION

An object of the present invention is to provide a novel and unobvious method and apparatus for pre-treating filter media.

Another object of a preferred embodiment of the present invention is to provide a method and apparatus for pre-treating filter media that overcomes at least one of the disadvantages of the prior art.

A further object of a preferred embodiment of the present invention is to eliminate or significantly reduce the costly and arduous procedures of on site backwashing, skimming and/or sterilizing filter media necessary to prepare the filter media for use in filter service runs.

Yet another object of a preferred embodiment of the present invention is to provide a method and apparatus that hydraulically removes fines and other unwanted material prior to installation of the filter media in a filter.

Still another object of a preferred embodiment of the present invention is to provide a method and apparatus that sterilizes filter media prior to installation of the filter media in a filter.

Yet still another object of a preferred embodiment of the present invention is to provide a method and apparatus that removes fines and other unwanted foreign materials from a lot of filter media while ensuring that the lot of filter media remains generally homogeneous.

It must be understood that no one embodiment of the present invention need include all of the aforementioned objects of the present invention. Rather, a given embodiment may include one or none of the aforementioned objects. Accordingly, these objects are not to be used to limit the scope of the claims of the present invention.

In summary, one embodiment of the present invention is directed to a method of pre-treating filter media used to filter water or wastewater prior to installation in a filter. The preferred method in accordance with this embodiment of the present invention includes the steps of: (a) sieving filter media to a predetermined size; (b) providing a pretreatment unit for temporarily storing and pre-treating filter media sized in said sieving step; (c) temporarily storing the filter media sized in the sieving step in the filter media pretreatment unit; and,

(c) washing the filter media temporarily stored in the pretreatment unit by directing fluid through the filter media pretreatment unit at a velocity sufficient to remove foreign material from the filter media, said washing step occurs prior to installation of the filter media in a filter.

Another embodiment of the present invention is directed to a method of pre-treating filter media used to filter water or wastewater prior to installation in a filter. The preferred method in accordance with this embodiment of the present invention includes the steps of: (a) sizing filter media to a predetermined size; (b) temporarily storing the filter media sized in the sizing step in a pretreatment unit for pre-treating filter media prior to installation in a filter; and, (c) treating the filter media temporarily stored in the pretreatment unit in at least one of the following manners: (i) washing the filter media while being temporarily stored in the pretreatment unit by directing liquid through the filter media at a velocity sufficient to remove fines and other unwanted materials from the filter media, the washing step occurs prior to installation of the filter media in a filter; and, (ii) sterilizing the filter media while being temporarily stored in the pretreatment unit.

A further embodiment of the present invention is directed to a pretreatment unit for pre-treating filter media used to filter water or wastewater prior to installation in a filter. The preferred treatment unit formed in accordance with this embodiment of the present invention includes a pretreatment chamber for temporarily storing and pre-treating filter media prior to installation of the filter media in a filter. The pretreatment chamber has a liquid distribution system for distributing liquid through the filter media at a sufficient velocity to remove fines or other unwanted material from the filter media. The pretreatment unit also includes a collector for collecting fines or other unwanted material removed from the filter media. The collector is operably associated with the pretreatment chamber. The pretreatment unit further includes a filter media discharge port for discharging filter media treated in the pretreatment chamber.

Still another embodiment of the present invention is directed to a method of pre-treating filter media used to filter water or wastewater prior to installation in a filter. The preferred method in accordance with this embodiment of the present invention includes the steps of: (a) sieving a first lot of filter media to a first size; (b) sieving a second lot of filter media to a second size, the first size being different from the second size; (c) providing a pretreatment unit for temporarily storing and pre-treating filter media sized in the sieving steps; (d) temporarily storing the first lot of filter media in the filter media pretreatment unit; (e) washing the first lot of filter media temporarily stored in the pretreatment unit by directing fluid through the filter media pretreatment unit at a velocity sufficient to remove foreign material from the filter media, the step of washing the first lot of filter media occurs prior to installation of the first lot of filter media in a filter; (f) temporarily storing the second lot of filter media in the filter media pretreatment unit; (g) washing the second lot of filter media temporarily stored in the pretreatment unit by directing fluid through the filter media pretreatment unit at a velocity sufficient to remove foreign material from the filter media, the step of washing the second lot of filter media occurs prior to installation of the second lot of filter media in a filter; (h) blending the first lot of filter media and second lot of filter media after the washing steps; and, (i) installing the filter media blended in the blending step in a filter.

Yet still another embodiment of the present invention is directed to a method of pre-treating filter media used to filter water or wastewater prior to installation in a filter. The preferred method in accordance with this embodiment of the present invention includes the steps of: (a) sizing filter media to a predetermined size; (b) temporarily storing the filter media sized in the sizing step in a pretreatment unit for pre-treating filter media prior to installation in a filter; (c) the washing the filter media while being temporarily stored in the pretreatment unit by directing liquid through the filter media at a sufficient velocity to fluidize the filter media to remove fines and other unwanted materials from the filter media, the washing step occurs prior to installation of the filter media in a filter; and, (d) subsequent to the washing step intermixing the filter media temporarily stored in the pretreatment unit to form a generally un-stratified and generally homogeneous lot of filter media, the step of intermixing occurs prior to installation of the filter media in a filter.

### BRIEF DESCRIPTION OF THE DRAWINGS

FIG. 1 is an elevation view of a pretreatment unit formed in accordance with a first preferred embodiment of the present invention with portions thereof omitted to better illustrate the inner workings of the pretreatment unit.

FIG. 2 is an elevation view of a pretreatment unit formed in accordance with a second preferred embodiment of the present invention with portions thereof omitted to better illustrate the inner workings of the pretreatment unit.

FIG. 3 is an elevation view of a pretreatment unit formed in accordance with a third preferred embodiment of the present invention with portions thereof omitted to better illustrate the inner workings of the pretreatment unit.

### DETAILED DESCRIPTION OF THE PREFERRED EMBODIMENTS OF THE INVENTION

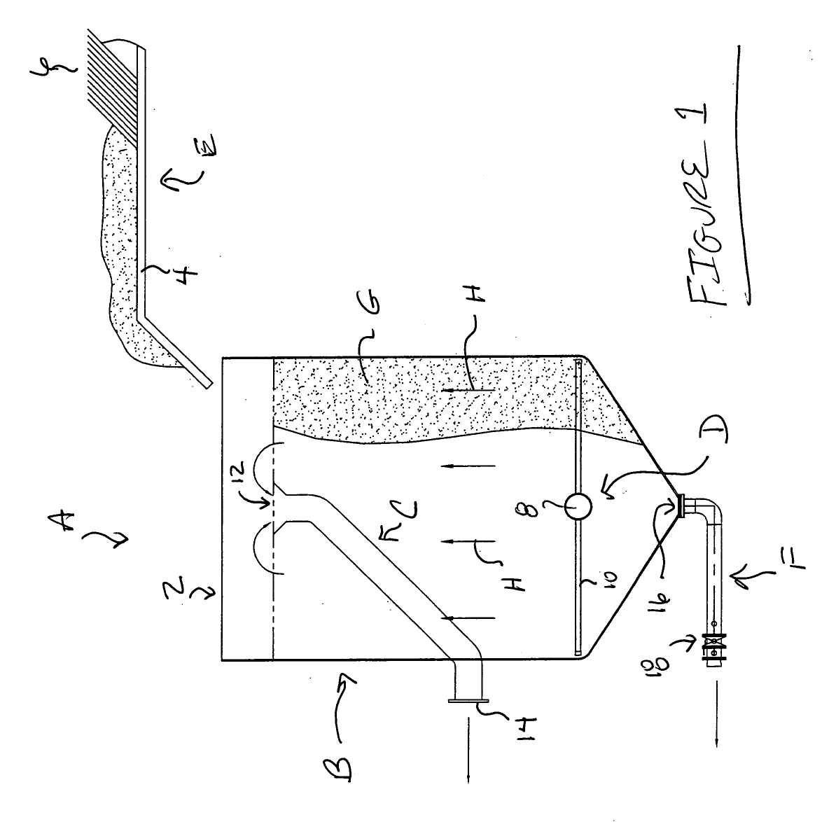

The preferred forms of the invention will now be described with reference to FIGS. 1-3. The appended claims are not limited to the preferred forms and no term and/or phrase used herein is to be given a meaning other than its ordinary meaning unless it is expressly stated that the term and/or phrase shall have a special meaning. As used herein the phrase “pretreatment unit” has a special meaning in that this phrase does not include within its meaning a filter for filtering water and/or wastewater including but not limited to a polishing filter and/or a roughing filter sometimes referred to as a clarifier, i.e., a filter for filtering water or wastewater is not a pretreatment unit. Further, the phrase “pretreatment unit” does not include a cyclone or any other device that relies on centrifugal force as the separating force.

### FIG. 1

Referring to FIG. 1, a portion of a pretreatment unit A is illustrated in one of many possible configurations. The pretreatment unit A includes a container B, a collector C, a fluid distribution system D, a filter media conveyor E and a filter media discharge conduit F. Preferably, container B temporarily stores the filter media G that has been mined and sized. The size and shape of container B may be varied as desired. For example, the container B may have a circular, square or rectangular cross-section. The top 2 of container B is preferably open so that the filter media G may be loaded in the container from above through the use of conveyor E or other suitable loading device. Preferably, conveyor E includes a filter media support 4 and a pushing element 6. However, it will be readily appreciated that the filter media G may be loaded into the container B in any other suitable manner.

The fluid distribution system D includes a main fluid supply pipe 8 operably connected to a fluid supply source (not shown) and a plurality of fluid distributors 10 (only one of which is shown) extending outwardly from supply pipe 8. The distributors 10 include a plurality of openings for directing the washing fluid upwardly through the filter media G. Arrows H indicate the direction of flow of the washing fluid. Preferably, the distributors 10 are evenly distributed along the longitudinal axis of the supply pipe 8 to ensure even and thorough distribution of the washing fluid throughout the filter media G temporarily stored in the container B. The washing fluid is pumped by any suitable means from the fluid supply source into the supply pipe 8 and distributors 10 where it is subsequently distributed through the filter media G.

The washing fluid is preferably filtered water. A sterilizing agent is preferably added to the washing fluid to sterilize the filter media G. Chlorine can be used as the sterilizing agent although any suitable sterilizing agent may be used. Preferably, the washing fluid is super chlorinated at chlorine levels ranging from approximately 25 ppm to approximately 100 ppm.

The collector C collects fines and other objectionable materials that are separated from the filter media during the washing process. Preferably, the inlet 12 of the collector C is centrally located in container B and extends slightly above the uppermost portion of the filter media G. Further, it is preferred that collector C extends approximately the depth of the container B. The outlet 14 of collector C is located outside of container B. Any suitable conduit may be connected to the outlet 14 of collector C to transport the materials and fluid passing through the collector C to a location for disposal or further treatment of the waste materials. It will be readily appreciated that the orientation and size of the collector C may be varied as desired.

Filter media discharge conduit F is connected to the outlet 16 of container B. Filter media discharge conduit F includes a valve 18 for controlling the flow of filter media through filter media discharge conduit F. When valve 18 is open, filter media is readily discharged from the container B where it can be inserted into sterilized containers, for example, sterilized plastic bags or sterilized bulk containers. In this manner, the present invention is able to eliminate or significantly reduce the likelihood that the filter media will become contaminated with unwanted foreign material after treatment in the pretreatment chamber up until installation of the filter media in a filter.

The preferred method of pretreatment will now be described. Initially, the filter media is mined and sized in any desired manner. The filter media may be sized into a number of lots of filter media of differing sizes. Each lot of filter media may be individually pretreated. Subsequently, two or more of the lots of filter media can be blended to form a desired size of filter media.

Once the filter media is installed in the container B, the washing fluid is pumped upwardly through the filter media temporarily stored in the container B. Preferably, the velocity of the washing fluid is sufficient to fluidize the filter media in the container B. Minimum fluidization velocity of the washing liquid v_{mf}, is determined by the following equation: v_{mf}=0.00381 (d_{60%})^{1.82} (sg−1)^{0.941}. ρ^{1.88}μ^{-0.88} where “d_{60%}” is the 60% size of the media particles in millimeters (equal to the product of the uniformity coefficient (UC) of the particles and the effective size of the particles (ES); “sg” is the specific gravity of the particles; “ρ” is the density of the liquid in lbs./cu.ft.; and “μ” is the viscosity of the liquid in centipoises. If the Reynolds number based on d_{60%} and V_{mf} is greater than 10, then the following multiplying factor must be applied:
K_{mf}=1.775Re_{mf}−0.272
where Re_{mf} is the Reynolds number based on d_{α%} and V_{mf}.

Fluidizing the filter media with an upward flow of washing fluid directs the unwanted foreign materials including the fines upwardly and into the collector C thereby hydraulically removing the unwanted foreign materials including the fines from the filter media. This hydraulic removal of fines is directly contrary to prior washing techniques employed in filtration systems. Specifically, numerous devices and arrangements have been devised to preclude the loss of filter media when the filter media is being washed in a filter. Once the unwanted foreign materials are hydraulically removed, the filter media in container B is allowed to soak in the washing liquid that has been super chlorinated. The sterilizing agent may be added to the washing fluid prior to entry into the container B or subsequent thereto. The sterilizing step is performed for a sufficient period of time to maintain a chlorine (disinfectant) residual for at least 24 hours until the disinfectant demand has stabilized. Once the filter media has been sterilized, it is dispensed from the container B into a suitable shipping container.

Where the velocity of the washing fluid is sufficient to fluidize the filter media, the filter media may settle in a stratified condition (i.e., larger particles on the bottom and smaller particles on the top) once the flow of the washing fluid is interrupted. In this instance, it will be desirable to intermix the filter media to achieve a generally homogeneous mixture of particles. This intermixing step can be performed by directing a fluid though the filter media G at velocities sufficient to intermix the filter media but not disrupt the filter media such that stratification occurs. The intermixing fluid can be air alone or concurrent air and water where the velocity of the water is less than the minimum fluidization velocity and most preferably less than ½ of the minimum fluidization velocity.

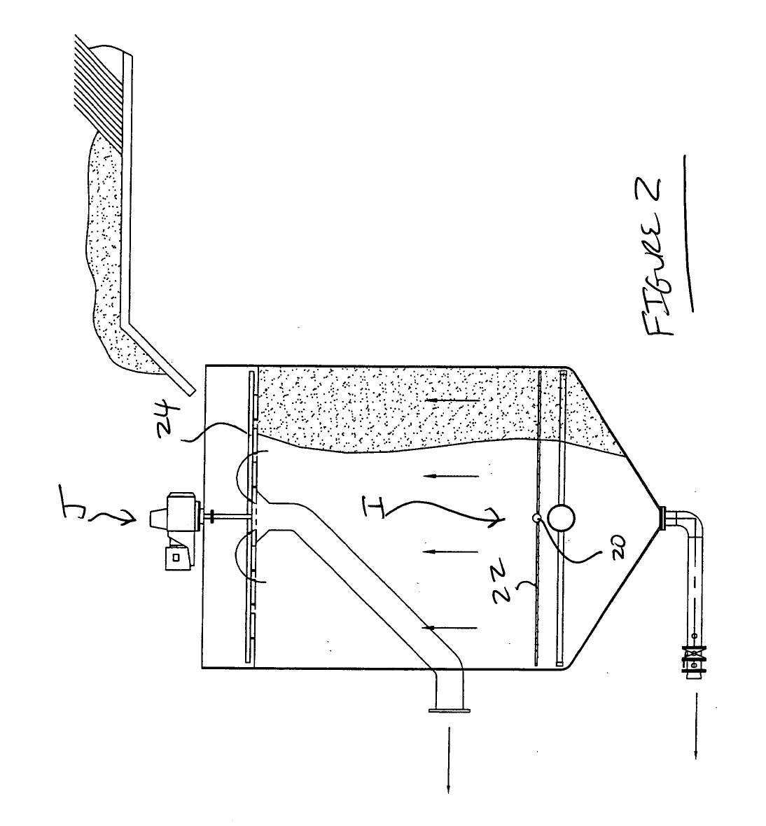

Additionally, air can be used to augment the backwash by introducing air into the filter media concurrent with water or by using just air. For example, where the density of the filter media or other circumstances make it impractical to provide a washing liquid at a sufficient velocity to fluidize the filter media, air can be introduced into container B concurrently with the washing fluid via air scour system I illustrated in FIG. 2 to assist in the removal of unwanted foreign materials from the filter media G. The air scour system I includes a main supply pipe 20 connected to the air source and a plurality of air distributors 22 (only one of which is shown). The air distributors 22 include a plurality of openings through which air is introduced into the filter media temporarily stored in container B. The simultaneous air scour/fluid wash is performed at sufficient rates to achieve collapse pulsing. This condition occurs when the velocity of the air and the washing liquid approximately fulfills the following relationship:
0.45(Qₐ)²+%v/v_{mf}=41.9
where “v” is the velocity of the backwash liquid, “v_{mf}” is the minimum fluidization velocity and “Qₐ” is the air scour rate in standard cubic feet per minute per square foot. Another example is where circumstances make it impractical to use liquid, the washing fluid can be air only.

The removal of unwanted foreign materials may also be augmented by a mechanical skimmer J including a rotating rake 24 as illustrated in FIG. 2. Preferably, the skimmer J is operated while the washing fluid is directed upwardly through the filter media.

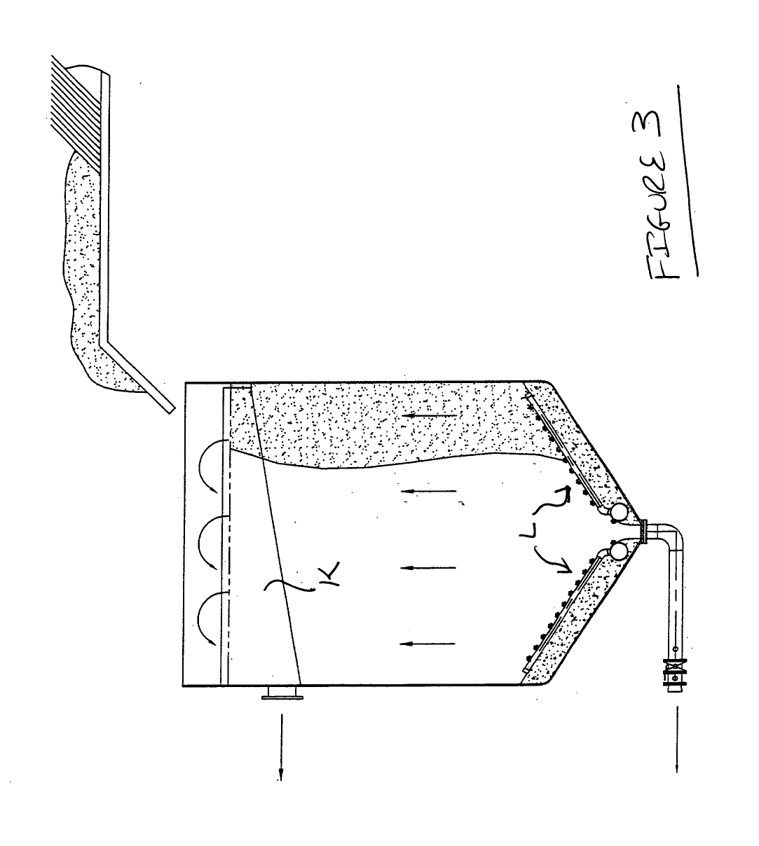

FIG. 3 depicts another alternative configuration for the pretreatment unit. In this embodiment, collector K and fluid distribution system L are configured slightly different than corresponding elements of the previously described embodiments. However, these elements operate in a similar fashion to the corresponding elements previously described.

While this invention has been described as having a preferred design, it is understood that the preferred design can be further modified or adapted following in general the principles of the invention and including but not limited to such departures from the present invention as come within the known or customary practice in the art to which the invention pertains. The claims are not limited to the preferred embodiment and have been written to preclude such a narrow construction using the principles of claim differentiation.

We claim:

1. A method of pre-treating filter media used to filter water or wastewater prior to installation in a filter, comprising the steps of:
(a) sieving filter media to a predetermined size;
(b) providing a pretreatment unit for temporarily storing and pre-treating filter media sized in said sieving step;
(c) temporarily storing the filter media sized in the sieving step in the filter media pretreatment unit; and,
(d) washing the filter media temporarily stored in the pretreatment unit by directing fluid through the filter media pretreatment unit at a velocity sufficient to remove foreign material from the filter media, said washing step occurs prior to installation of the filter media in a filter.

2. A method as recited in claim 1, wherein:
(a) the washing fluid is a liquid and the velocity of the liquid is sufficient to fluidize the filter media temporarily stored in the pretreatment unit to remove fines and other unwanted foreign material from the filter media.

3. A method as recited in claim 2, including the further steps of:
(a) providing a foreign material collector for collecting fines and other unwanted material removed from the filter media during said washing step, the foreign material collector having an inlet and an outlet; and,
(b) positioning the foreign material collector such that the inlet of the foreign material collector is located above the top of the filter media temporarily stored in the pretreatment unit.

4. A method as recited in claim 1, wherein:
(a) the washing fluid in the washing step is concurrent air and liquid.

5. A method as recited in claim 4, wherein:
(a) the air and liquid is directed through the filter media in said washing step at rates that result in collapse pulsing of the filter media.

6. A method as recited in claim 1, including the further steps of:
(a) sterilizing the filter media temporarily stored in the pretreatment unit; and,
(b) conveying the filter media sterilized during the sterilizing step from the pretreatment unit to a sterilized container.

7. A method as recited in claim 6, wherein:
(a) said sterilizing step includes soaking the filter media in a chlorinated liquid.

8. A method as recited in claim 7, including the further step of:
(a) providing a chlorinated liquid in the sterilized container having filter media pretreated by the pretreatment unit.

9. A method as recited in claim 8, including the further step of:
(a) transporting the sterilized container to the site of the filter subsequent to the providing step recited in paragraph (a) in claim 8.

10. A method of pre-treating filter media used to filter water or wastewater prior to installation in a filter, comprising the steps of:
(a) sizing filter media to a predetermined size;
(b) temporarily storing the filter media sized in said sizing step in a pretreatment unit for pre-treating filter media prior to installation in a filter; and,
(c) treating the filter media temporarily stored in the pretreatment unit in at least one of the following manners:
(i) washing the filter media while being temporarily stored in the pretreatment unit by directing liquid through the filter media at a velocity sufficient to remove fines and other unwanted materials from the filter media, said washing step occurs prior to installation of the filter media in a filter; and,
(ii) sterilizing the filter media while being temporarily stored in the pretreatment unit.

11. A method as recited in claim 10, including the further step of:
(a) dispensing the filter media treated in said treating step from the pretreatment unit.

12. A method as recited in claim 11, including the further step of:
(a) installing the filter media dispensed during said dispensing step into a filter.

13. A method as recited in claim 10, wherein:
(a) said step of treating includes both washing and sterilizing the filter media while temporarily stored in the pretreatment unit.

14. A method as recited in claim 13, wherein:
(a) the washing fluid is a liquid and the velocity of the liquid is sufficient to fluidize the filter media.

15. A pretreatment unit for pre-treating filter media used to filter water or wastewater prior to installation in a filter, comprising:
(a) a pretreatment chamber for temporarily storing and pre-treating filter media prior to installation of the filter media in a filter; said pretreatment chamber having a liquid distribution system for distributing liquid through the filter media at a sufficient velocity to remove fines or other unwanted material from the filter media;
(b) a collector for collecting fines or other unwanted material removed from the filter media, said collector being operably associated with said pretreatment chamber; and,
(c) a filter media discharge port for discharging filter media treated in the pretreatment chamber.

16. An apparatus as set forth in claim 15, further including:
(a) a transport device for transporting sized filter media to said pretreatment chamber.

17. An apparatus as set forth in claim 15, wherein:
(a) said collector includes an inlet and an outlet, said inlet of said collector is disposed above the top of the filter media temporarily stored in said pretreatment chamber.

18. An apparatus as set forth in claim 15, wherein:
(a) at least a portion of said collector is disposed in said pretreatment chamber.

19. An apparatus as set forth in claim 15, wherein:
(a) said pretreatment chamber further includes an air scour system for scouring the filter media temporarily stored in the pretreatment chamber.

20. An apparatus as set forth in claim 15, wherein:
(a) said pretreatment chamber further includes a skimmer for assisting in the removal of fines from the filter media temporarily stored in the pretreatment chamber.

21. A method of pre-treating filter media used to filter water or wastewater prior to installation in a filter, comprising the steps of:
(a) sieving a first lot of filter media to a first size;
(b) sieving a second lot of filter media to a second size, the first size being different from the second size;
(c) providing a pretreatment unit for temporarily storing and pre-treating filter media sized in said sieving steps;
(d) temporarily storing the first lot of filter media in the filter media pretreatment unit;
(e) washing the first lot of filter media temporarily stored in the pretreatment unit by directing fluid through the filter media pretreatment unit at a velocity sufficient to remove foreign material from the filter media, said step of washing the first lot of filter media occurs prior to installation of the first lot of filter media in a filter;
(f) temporarily storing the second lot of filter media in the filter media pretreatment unit;
(g) washing the second lot of filter media temporarily stored in the pretreatment unit by directing fluid through the filter media pretreatment unit at a velocity sufficient to remove foreign material from the filter media, said step of washing the second lot of filter media occurs prior to installation of the second lot of filter media in a filter;
(h) blending the first lot of filter media and second lot of filter media after the washing steps; and,
(i) installing the filter media blended in the blending step in a filter.

22. A method of pre-treating filter media used to filter water or wastewater prior to installation in a filter, comprising the steps of:
(a) sizing filter media to a predetermined size;
(b) temporarily storing the filter media sized in said sizing step in a pretreatment unit for pre-treating filter media prior to installation in a filter;
(c) washing the filter media while being temporarily stored in the pretreatment unit by directing liquid through the filter media at a sufficient velocity to fluidize the filter media to remove fines and other unwanted materials from the filter media, said washing step occurs prior to installation of the filter media in a filter; and,
(d) subsequent to said washing step intermixing the filter media temporarily stored in the pretreatment unit to form a generally un-stratified and generally homogeneous lot of filter media, said step of intermixing occurs prior to installation of the filter media in a filter.

23. A method as recited in claim 22, wherein:
(a) the intermixing step includes directing a fluid upwardly through the filter media at a sufficiently low rate to form a generally un-stratified and generally homogeneous lot of filter media

24. A method as recited in claim 23, wherein:
(a) the intermixing step includes directing a combination of liquid and air upwardly through the filter media at a sufficiently low rate to form a generally un-stratified and generally homogeneous lot of filter media, the velocity of the liquid is less than the minimum fluidization velocity.

25. A method as recited in claim 22, wherein:
(a) the intermixing step includes directing air only upwardly through the filter media at a sufficiently low rate to form a generally un-stratified and generally homogeneous lot of filter media.
